(19) United States
(12) Patent Application Publication
(10) Pub. No.: US 2016/0346520 A1
(43) Pub. Date: Dec. 1, 2016

(54) Medical Instrument for Injecting an Agent

(71) Applicant: Dan B. French, Dallas, TX (US)

(72) Inventor: Dan B. French, Dallas, TX (US)

(21) Appl. No.: 14/809,938

(22) Filed: Jul. 27, 2015

Related U.S. Application Data
(60) Provisional application No. 62/167,681, filed on May 28, 2015.

(51) Int. Cl. A61M 25/10; A61M 5/19; A61M 1/00

(52) U.S. Cl. CPC A61M 25/10; A61M 1/008; A61M 5/19; A61M 2025/1061; A61M 2210/1092

(57) ABSTRACT

A medical instrument for injecting an agent is disclosed. In one embodiment, a main elongated cylindrical body has a multiple channels therethrough that house a balloon inflation tube and two syringe push tubes having injection needles. Each of the syringe push tubes exit at respective lateral exit ports positioned on opposite sides of the main elongated cylindrical body. A secondary elongated cylindrical body is coupled to the main cylindrical body and includes a retention balloon positioned in fluid communication with the balloon inflation tube. During operation, the retention balloon is filled with liquid through to act as a retainer for the medical instrument inside a bladder of a patient to position and immobilize the medical instrument inserted into a sphincter of a urethra of the patient. During this process, the injection needles are deployed and the agent, such as a bulking agent, is injected.

### PRIORITY STATEMENT & CROSS-REFERENCE TO RELATED APPLICATIONS

This application claims priority from U.S. patent application Ser. No. 62/167,681 entitled “Medical Instrument for Injecting an Agent” and filed on May 28, 2015, in the name of Dan B. French; which is hereby incorporated by reference for all purposes.

### TECHNICAL FIELD OF THE INVENTION

This invention relates, in general, to body inserted tubular conduit structures and, in particular, to medical instruments for injecting an agent, such as a bulking agent, for use in treatment of urinary incontinence in women, for example.

### BACKGROUND OF THE INVENTION

Stress urinary incontinence (SUI) is a common problem affecting many women. Estimates vary, but it is probably reasonable to assume that SUI affects 15-25% of women in the United States and the prevalence increases with age. Though there are several different treatment options for SUI, injection of urethral bulking agents is an established, accepted, minimally invasive form of treatment for mild to moderate SUI. Currently delivery of approved urethral bulking agents into the urethra requires a cystoscope and the requisite equipment to place the material under direct vision, making in-office application feasible only for those physicians with a cystoscope. There is an existing need for apparatuses and techniques that do not require a cystoscope for application of urethral bulking agents.

### SUMMARY OF THE INVENTION

It would be advantageous to develop apparatuses and techniques that do not require a cystoscope for the application of urethral bulking agents. It would also be desirable to enable a mechanical-based solution that would provide a medical instrument for injecting an agent in a safe and efficient manner. To better address one or more of these concerns, a medical instrument for injecting an agent, such a urethral bulking agent, is disclosed. In one embodiment, a main elongated cylindrical body has a multiple channels therethrough that house a balloon inflation tube and two syringe push tubes having injection needles. Each of the syringe push tubes exit at respective lateral exit ports positioned on opposite sides of the main elongated cylindrical body. A secondary elongated cylindrical body is coupled to the main cylindrical body and includes a retention balloon positioned in fluid communication with the balloon inflation tube. During operation, the retention balloon is filled with liquid through to act as a retainer for the medical instrument inside a bladder of a patient to position and immobilize the medical instrument inserted into a sphincter of a urethra of the patient. During this process, the injection needles are deployed and the agent, such as a bulking agent, is injected and two separate target locations receive the bulking agent treatment. These and other aspects of the invention will be apparent from and elucidated with reference to the embodiments described hereinafter.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a front elevation view of one embodiment of a medical instrument for injecting an agent according to the teachings presented herein;

FIG. 2 is a front elevation view of the medical instrument depicted in FIG. 1 having enlarged illustrated detail;

FIG. 3 is a cross-sectional view of the medical instrument along line 3-3 of FIG. 2;

FIG. 4 is a cross-sectional view of the medical instrument along line 4-4 of FIG. 2;

FIG. 5 is a cross-sectional view of the medical instrument along line 5-5 of FIG. 2;

FIG. 6 is a simplified anatomical sketch to show a first mode of application of the medical instrument;

FIG. 7 is a simplified anatomical sketch to show a second mode of application of the medical instrument; and

FIG. 8 is a simplified anatomical sketch to show a third mode of application of the medical instrument.

### DETAILED DESCRIPTION OF THE INVENTION

While the making and using of various embodiments of the present invention are discussed in detail below, it should be appreciated that the present invention provides many applicable inventive concepts, which can be embodied in a wide variety of specific contexts. The specific embodiments discussed herein are merely illustrative of specific ways to make and use the invention, and do not delimit the scope of the present invention.

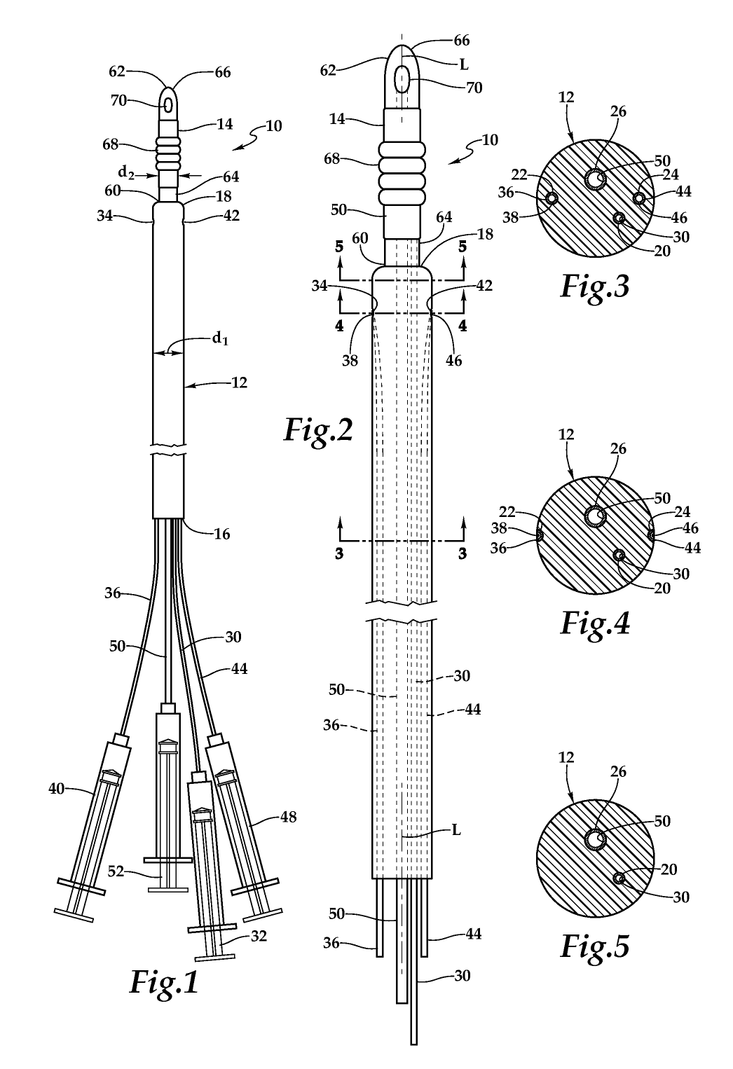

Referring initially to FIG. 1 through FIG. 5, therein is depicted one embodiment of a medical instrument which is schematically illustrated and designated 10. The medical instrument 10 includes a main elongated body 12 and a secondary elongated body 14. The main elongated cylindrical body 12 has a diameter d1 and includes a distal end 16 and a proximal end 18. A longitudinal axis L runs from the distal end 16 to the proximal end 18. Channels 20, 22, 24, 26 traverse the main elongated cylindrical body 12. The channel 20 houses a balloon inflation tube 30 therein. As shown, the channel 20 traverses the main elongated cylindrical body such that the balloon inflation tube 30 enters the secondary elongated body 14 to establish fluid communication therewith. A syringe 32 is positioned at the end of the balloon inflation tube 30. During operation, the syringe may deliver an appropriate pre-determined amount of fluid to the secondary elongated body 14 as discussed in more detail hereinbelow.

The channel 22 traverses the main elongated cylindrical body from the distal end 16 to a lateral exit port 34 near the proximal end 18. The channel 22 houses a syringe push tube 34 having an injection needle 38 at the end thereof. At the other end, a syringe 40 is positioned to deliver an appropriate amount of bulking agent during use. Similarly, the channel 24 traverses the main elongated cylindrical body 12 from the distal end 16 to a lateral exit port 42 near the proximal end 18. The channel 24 houses a syringe push tube 44 having an injection needle 46 at the end thereof and a syringe 48 holding a supply of bulking agent, for example, at the opposite end. As illustrated, the lateral exit port 42 positioned on the main elongated cylindrical body 12 is substantially opposite the lateral exit port 34.

As mentioned, the syringe push tube 36 includes the injection needle 38, which has a retracted position wherein the injection needle 38 is within the channel 22 and a deployed position wherein the injection needle 38 is exterior to the channel 22 forming an acute angle with the longitudinal axis L. Likewise, the injection needle 46 has a retracted position wherein the injection needle 46 is within the channel 24 and a deployed position wherein the injection needle 46 is exterior to the third channel 24.

The channel 26 also traverses the main elongated cylindrical body 12 and enters the secondary elongated cylindrical body 14 to establish fluid communication therewith. The channel 25 houses a drainage tube 50 having a syringe 52 located at the end thereof. With particular reference to FIG. 3 through FIG. 5, in one embodiment, the main elongated cylindrical body 12 may be a solid core body having the channels 20, 22, 24, 26 bored therethrough. The first elongated cylindrical body may be a size of approximately 6 mm in diameter or the size of approximately a No. 18 French catheter.

The secondary elongated cylindrical body 14 includes a distal end 60 and a proximal end 62 such that the distal end 60 of secondary elongated cylindrical body 14 is coupled to proximal end 18 of the main cylindrical body 12. The secondary elongated cylindrical body 14 has a diameter d2, which is less than the diameter d1 of the main elongated cylindrical body 12, and the secondary elongated cylindrical body 14 is aligned with the longitudinal axis L.

The secondary elongated cylindrical body 14 includes a bladder neck 64 at the distal end 60 and an insertion tip 66 at the proximal end 62. The second elongated cylindrical body 14 includes a retention balloon 68 secured about the second elongated cylindrical body and interposed between the bladder neck 64 and the insertion tip 66. As shown, in one implementation, the retention balloon 68 is of a predetermined size made of a soft and flexible material. A drainage opening 70 is located proximate to the insertion tip 66 to provide fluid communication between a bladder of a patient and the syringe 52. In one embodiment, the second elongated cylindrical body 14 may be a size of approximately 4 mm in diameter or a size of approximately a No. 12 French catheter. When considered in combination, in one implementation, the main elongated cylindrical body 12 and the secondary elongated cylindrical body 14 may comprise a length of approximately 203 mm or 8 inches.

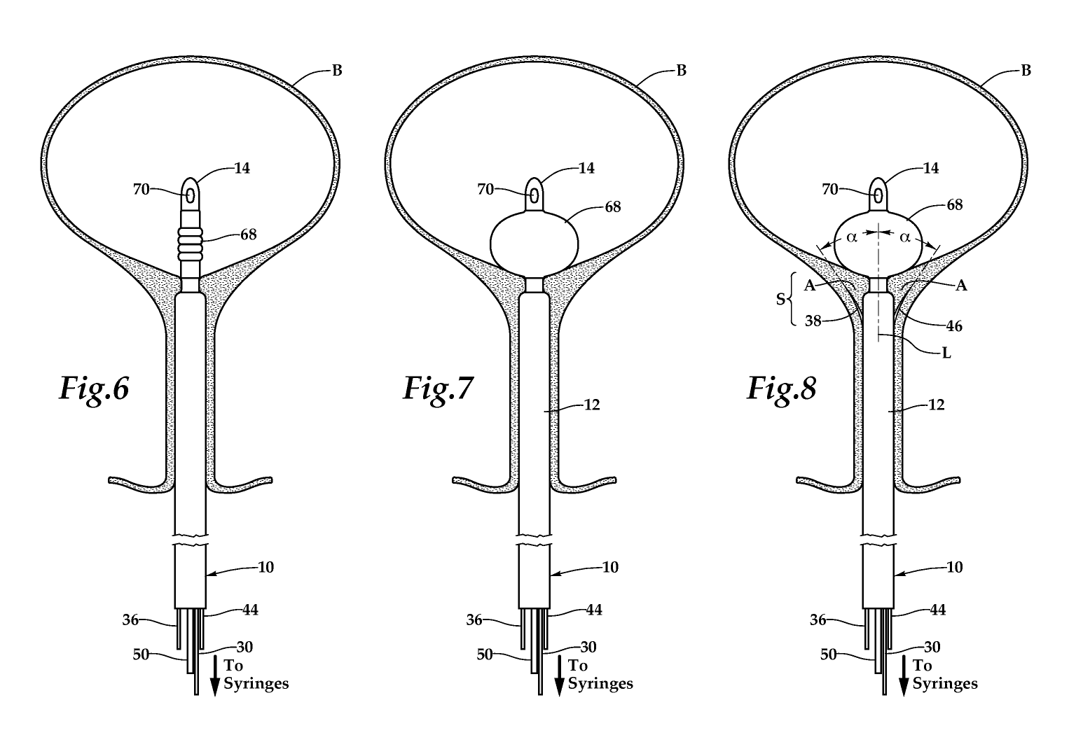

Referring now to FIG. 6 through FIG. 8, the medical instrument 10 is inserted inside a bladder B of a patient. Thereafter, the retention balloon 68 is filled with liquid via the balloon inflation tube 30 and syringe 32 in order to act, in its inflated position, as a retainer for the medical instrument 10 inside the bladder B of the patient. As shown, upon the inflation of the retention balloon 68, the medical instrument is positioned and immobilized at a sphincter of a urethra of the patient. During this process, the injection needles 38, 46 are deployed to inject the agent, such as bulking agent A. The bulking agent A enters a tissue of the sphincter at a correct location and to a correct depth and angle. Further, the bulking agent A may be substantially simultaneously applied to two separate locations at the sphincter to increase efficacy of the treatment.

As shown, the injection needles 38, 46 are deployed in a gradual, non-deviated exit at the lateral exit ports 34, 42. With respect to the injection needles 38, 46, a deployed length may be approximately 10 mm to approximately 35 mm. A deployed length between approximately 12 mm to approximately 22 mm is also suitable. Suitable still is a deployed length of approximately 15 mm to approximately 18 mm.

As illustrated, the distance from the bladder neck to each of the deployed injection needles 38, 46 may comprise a distance of approximately 3 mm to approximately 30 mm, as shown by separation distance S. A suitable separation distance S is also from approximately 4 mm to approximately 15 mm. Further suitable is a separation distance S of approximately 8 mm. With respect to the deployed length of the injection needles 38, 46, a deployed length of approximately 10 mm to approximately 35 mm is suitable. A deployed length of approximately 12 mm to approximately 22 mm is also suitable and a deployed length of approximately 15 mm to approximately 18 mm is also suitable. The injection needles 38, 46 have deployed angle, alpha, with respect to the longitudinal axis L of approximately 10 degrees to approximately 45 degrees, in one embodiment. A deployed angle, alpha α, with the longitudinal axis of approximately 20 degrees to approximately 40 degrees is suitable. Further suitable is a deployed angle, alpha α, with the longitudinal axis L of approximately 25 degrees to approximately 30 degrees.

The order of execution or performance of the methods and procedural flows illustrated and described herein is not essential, unless otherwise specified. That is, elements of the methods and procedural flows may be performed in any order, unless otherwise specified, and that the methods may include more or less elements than those disclosed herein. For example, it is contemplated that executing or performing a particular element before, contemporaneously with, or after another element are all possible sequences of execution.

While this invention has been described with reference to illustrative embodiments, this description is not intended to be construed in a limiting sense. Various modifications and combinations of the illustrative embodiments as well as other embodiments of the invention, will be apparent to persons skilled in the art upon reference to the description. It is, therefore, intended that the appended claims encompass any such modifications or embodiments.

What is claimed is:

1. A medical instrument for injecting an agent, comprising:
a first elongated cylindrical body having a first distal end and a first proximal end, the first elongated cylindrical body having a first diameter and a longitudinal axis from the first distal end to the first proximal end, the first elongated cylindrical body having a plurality of channels therethrough;
a second elongated cylindrical body having a distal end and a proximal end, the distal end of second cylindrical body being coupled to proximal end of the first cylindrical body;
the second elongated cylindrical body having a second diameter which is less than the first diameter of the first elongated body, the second elongated cylindrical body being aligned with the longitudinal axis;
the second elongated cylindrical body having a bladder neck at the distal end and an insertion tip at the proximal end, the second elongated cylindrical body having a retention balloon secured about the second elongated cylindrical body and interposed between the bladder neck and the insertion tip, the retention balloon being of a predetermined size made of a soft and flexible material;
the plurality of channels including a first channel housing a balloon inflation tube therein, the first channel traversing the first elongated cylindrical body and entering the second elongated cylindrical body to establish fluid communication with the retention balloon;
the plurality of channels including a second channel housing a first syringe push tube, the second channel traversing the first elongated cylindrical body from the first distal end to a first lateral exit port proximate the first proximal end;
the plurality of channels including a third channel housing a second syringe push tube, the third channel traversing the first elongated cylindrical body from the first distal end to a second lateral exit port proximate the first proximal end, the second lateral exit port being positioned on the first elongated cylindrical body substantially opposite the first lateral exit port;
the first syringe push tube including a first injection needle having a retracted position wherein the first injection needle is within the second channel and a deployed position wherein the first injection needle is exterior to the second channel forming an acute angle with the longitudinal axis; and
the second syringe push tube including a second injection needle having a retracted position wherein the second injection needle is within the third channel and a deployed position wherein the second injection needle is exterior to the third channel forming an acute angle with the longitudinal axis,
whereby the retention balloon filled with liquid through the balloon inflated tube acts as a retainer for the medical instrument inside a bladder of a patient, positioning and immobilizing the medical instrument inserted into a sphincter of a urethra of the patient, during which process the first injection needle is deployed to inject the agent, during which process the second injection needle is deployed to inject the agent, thereby treating two locations at the sphincter.

2. The medical instrument as recited in claim 1, wherein the first and second injection needles are deployed in a gradual, non-deviated exit.

3. The medical instrument as recited in claim 1, wherein the plurality of channels further comprises a fourth channel housing a drainage tube therein, the fourth channel traversing the first elongated cylindrical body and entering the second elongated cylindrical body to establish fluid communication with a drainage port within the second elongated cylindrical body, the drainage port being proximate the insertion tip.

4. The medical instrument as recited in claim 1, wherein the first elongated cylindrical body further comprises a solid core body having the plurality of channels bored therethrough.

5. The medical instrument as recited in claim 1, wherein the first elongated cylindrical body further comprises a size of approximately 6 mm in diameter.

6. The medical instrument as recited in claim 1, wherein the first elongated cylindrical body further comprises a size of approximately a No. 18 French catheter.

7. The medical instrument as recited in claim 1, wherein the second elongated cylindrical body further comprises a size of approximately 4 mm in diameter.

8. The medical instrument as recited in claim 1, wherein the second elongated cylindrical body further comprises a size of approximately a No. 12 French catheter.

9. The medical instrument as recited in claim 1, wherein the first and second elongated cylindrical bodies, in combination, comprise a length of approximately 203 mm.

10. The medical instrument as recited in claim 1, wherein the distance from the bladder neck to each of the deployed first and second injection needles comprises a distance from approximately 3 mm to approximately 30 mm.

11. The medical instrument as recited in claim 1, wherein the distance from the bladder neck to each of the deployed first and second injection needles comprises a distance from approximately 4 mm to approximately 15 mm.

12. The medical instrument as recited in claim 1, wherein the distance from the bladder neck to each of the deployed first and second injection needles comprises a distance of approximately 8 mm.

13. The medical instrument as recited in claim 1, wherein each of the first and second injection needles comprises a deployed length of approximately 10 mm to approximately 35 mm.

14. The medical instrument as recited in claim 1, wherein each of the first and second injection needles comprises a deployed length of approximately 12 mm to approximately 22 mm.

15. The medical instrument as recited in claim 1, wherein each of the first and second injection needles comprises a deployed length of approximately 15 mm to approximately 18 mm.

16. The medical instrument as recited in claim 1, wherein each of the first and second injection needles comprises a deployed angle with the longitudinal axis of approximately 10 degrees to approximately 45 degrees.

17. The medical instrument as recited in claim 1, wherein each of the first and second injection needles comprises a deployed angle with the longitudinal axis of approximately 20 degrees to approximately 40 degrees.

18. The medical instrument as recited in claim 1, wherein each of the first and second injection needles comprises a deployed angle with the longitudinal axis of approximately 25 degrees to approximately 30 degrees.

19. A medical instrument for injecting an agent, comprising:
a first elongated cylindrical body having a first distal end and a first proximal end, the first elongated cylindrical body having a first diameter and a longitudinal axis from the first distal end to the first proximal end, the first elongated cylindrical body having a plurality of channels therethrough;
a second elongated cylindrical body having a distal end and a proximal end, the distal end of second cylindrical body being coupled to proximal end of the first cylindrical body;
the second elongated cylindrical body having a second diameter which is less than the first diameter of the first elongated body, the second elongated cylindrical body being aligned with the longitudinal axis;
the second elongated cylindrical body having a bladder neck at the distal end and an insertion tip at the proximal end, the second elongated cylindrical body having a retention balloon secured about the second elongated cylindrical body and interposed between the bladder neck and the insertion tip, the retention balloon being of a predetermined size made of a soft and flexible material;
the second elongated cylindrical body having a drainage port proximate the insertion tip;
the plurality of channels including a first channel housing a balloon inflation tube therein, the first channel traversing the first elongated cylindrical body and entering the second elongated cylindrical body to establish fluid communication with the retention balloon;
the plurality of channels including a second channel housing a first syringe push tube, the second channel traversing the first elongated cylindrical body from the first distal end to a first lateral exit port proximate the first proximal end;
the plurality of channels including a third channel housing a second syringe push tube, the third channel traversing the first elongated cylindrical body from the first distal end to a second lateral exit port proximate the first proximal end, the second lateral exit port being positioned on the first elongated cylindrical body substantially opposite the first lateral exit port;
the plurality of channels including a fourth channel housing a drainage tube therein, the fourth channel traversing the first elongated cylindrical body and entering the second elongated cylindrical body to establish fluid communication with the drainage port within the second elongated cylindrical body;
the first syringe push tube including a first injection needle having a retracted position wherein the first injection needle is within the second channel and a deployed position wherein the first injection needle is exterior to the second channel forming an acute angle with the longitudinal axis; and
the second syringe push tube including a second injection needle having a retracted position wherein the second injection needle is within the third channel and a deployed position wherein the second injection needle is exterior to the third channel forming an acute angle with the longitudinal axis,
whereby the retention balloon filled with liquid through the balloon inflated tube acts as a retainer for the medical instrument inside a bladder of a patient, positioning and immobilizing the medical instrument inserted into a sphincter of a urethra of the patient, during which process the first injection needle is deployed in a gradual, non-deviated exit to inject the agent, during which process the second injection needle is deployed in a gradual, non-deviated exit to inject the agent, thereby treating two locations at the sphincter.

20. A medical instrument for injecting an agent, comprising:
a first elongated cylindrical body having a first distal end and a first proximal end, the first elongated cylindrical body having a first diameter and a longitudinal axis from the first distal end to the first proximal end, the first elongated cylindrical body having a plurality of channels therethrough;
a second elongated cylindrical body having a distal end and a proximal end, the distal end of second cylindrical body being coupled to proximal end of the first cylindrical body;
the second elongated cylindrical body having a second diameter which is less than the first diameter of the first elongated body, the second elongated cylindrical body being aligned with the longitudinal axis;
the second elongated cylindrical body having a bladder neck at the distal end and an insertion tip at the proximal end, the second elongated cylindrical body having a retention balloon secured about the second elongated cylindrical body and interposed between the bladder neck and the insertion tip, the retention balloon being of a predetermined size made of a soft and flexible material;
the second elongated cylindrical body having a drainage port proximate the insertion tip;
the plurality of channels including a first channel housing a balloon inflation tube therein, the first channel traversing the first elongated cylindrical body and entering the second elongated cylindrical body to establish fluid communication with the retention balloon;
the plurality of channels including a second channel housing a first syringe push tube, the second channel traversing the first elongated cylindrical body from the first distal end to a first lateral exit port proximate the first proximal end;
the plurality of channels including a third channel housing a second syringe push tube, the third channel traversing the first elongated cylindrical body from the first distal end to a second lateral exit port proximate the first proximal end, the second lateral exit port being positioned on the first elongated cylindrical body substantially opposite the first lateral exit port;
the plurality of channels including a fourth channel housing a drainage tube therein, the fourth channel traversing the first elongated cylindrical body and entering the second elongated cylindrical body to establish fluid communication with the drainage port within the second elongated cylindrical body;
the first syringe push tube including a first injection needle having a retracted position wherein the first injection needle is within the second channel and a deployed position wherein the first injection needle is exterior to the second channel forming an acute angle of between approximately 20 degrees and approximately 40 degrees with the longitudinal axis;
the second syringe push tube including a second injection needle having a retracted position wherein the second injection needle is within the third channel and a deployed position wherein the second injection needle is exterior to the third channel forming an acute angle of between approximately 20 degrees and approximately 40 degrees with the longitudinal axis; and
the first and second injection needles, upon deployment, being between approximately 4 mm and approximately 15 mm from the bladder neck,
whereby the retention balloon filled with liquid through the balloon inflated tube acts as a retainer for the medical instrument inside a bladder of a patient, positioning and immobilizing the medical instrument inserted into a sphincter of a urethra of the patient, during which process the first injection needle is deployed in a gradual, non-deviated exit to inject the agent, during which process the second injection needle is deployed in a gradual, non-deviated exit to inject the agent, thereby treating two locations at the sphincter.
